(19) United States
(12) Patent Application Publication
(10) Pub. No.: US 2025/0082306 A1
(43) Pub. Date: Mar. 13, 2025

(54) APPARATUS, METHOD AND COMPUTER READABLE MEDIA TO CONTROL AN ULTRASOUND IMAGING SYSTEM BASED ON CONTEXT PARAMETERS OF AN ULTRASOUND IMAGING SESSION

(71) Applicant: Exo Imaging, Inc., Santa Clara, CA (US)

(72) Inventors: Seshadri Srinivasan, Fremont, CA (US); Biren J. Parmar, San Jose, CA (US); Ruiying Zhang, San Jose, CA (US); John Matthew Weston, El Granada, CA (US)

(73) Assignee: Exo Imaging, Inc., Santa Clara, CA (US)

(21) Appl. No.: 18/466,355

(22) Filed: Sep. 13, 2023

(51) Int. Cl. A61B 8/00; G16H 10/60

(52) U.S. Cl. CPC A61B 8/54; A61B 8/565; G16H 10/60

(57) ABSTRACT

An ultrasound imaging system, a method, and computer-implemented media. The system includes: context determination circuitry (CDC) to generate context signals based on one or more ultrasound imaging session contexts of an ultrasound imaging session; a memory storing a context information engine (CIE); circuitry coupled to the memory and adapted to execute the CIE to determine one or more context parameters of the ultrasound imaging session based on the context signals from the CDC; and processing circuitry to: access the one or more context parameters before or during the ultrasound imaging session; and determine one or more ultrasound imaging settings of the ultrasound imaging session based on the one or more context parameters; and cause the one or more settings to be implemented for the ultrasound imaging session.

### FIELD

Embodiments relate in general to the field of ultrasound imaging systems.

### BACKGROUND

Ultrasound imaging is widely used in the fields of medicine and non-destructive testing and may have a diagnostic or a procedural purpose. An ultrasound imaging system, such as an ultrasound imaging probe, may operate based on certain settings, such as various presets, imaging modes, frequencies, gains and/or associated imaging frame rates. The versatility of an ultrasound imaging system is made possible in part by advances with respect to its settings has made the need for preset optimization even more significant.

### SUMMARY

A circuitry may be configured to determine and cause implementation of presets of an imaging session by an ultrasound imaging system. The circuitry may be separate from or part of the ultrasound imaging system. The circuitry may use one or more context parameters, such as those based on light, sound, location, or time, in order to implement one or more ultrasound imaging settings relating to the imaging session. The implemented one or more settings may correspond to an imaging session preset. The circuitry may cause to be stored, in a memory circuitry, a mapping of the one or more context parameters to the one or more settings.

### BRIEF DESCRIPTION OF THE DRAWINGS

The novel features of embodiments are set forth with particularity in the appended claims. A better understanding of the features and advantages of Some embodiments will be obtained by reference to the following detailed description that sets forth illustrative embodiments, in which the principles of the invention are utilized, and the accompanying drawings (also “Figure” and “FIG.” herein), of which:

FIG. 1 is a block diagram of an ultrasound imaging device in accordance with some embodiments.

FIG. 2 is a diagram of an ultrasound imaging network in accordance with some embodiments.

FIG. 3 is a schematic diagram of an ultrasound imaging device in accordance with some embodiments.

FIG. 4 depicts a flowchart of a process according to a first embodiment.

### DETAILED DESCRIPTION

Some embodiments advantageously provide an ultrasound imaging system, such as an ultrasound imaging probe or a computing device paired to the probe and adapted to display ultrasound images corresponding to an ultrasound imaging session performed using the probe. An ultrasound imaging system includes: context determination circuitry (CDC) (e.g. a sensor, a clock, a communication circuitry to determine context from received signals) to generate context signals based on one or more ultrasound imaging session contexts (e.g., light, sound, time, location) of an ultrasound imaging session; a memory storing a context information engine (CIE) (e.g., software that takes the context signals, and generates context parameters that are useful for a determination of settings or presets); circuitry (e.g., could be a dedicated circuitry or part of any existing processor on the system) coupled to the memory and adapted to execute the CIE to determine one or more context parameters of the ultrasound imaging session based on the context signals from the CDC; and processing circuitry to: access the one or more context parameters before or during (e.g., processing circuitry could receive updated/new context parameters while an imaging session is taking place) the ultrasound imaging session; and determine one or more ultrasound imaging settings (e.g., a setting, a group of settings, a preset (that includes a group of settings)) of the ultrasound imaging session based on the one or more context parameters; and cause the one or more settings to be implemented for the ultrasound imaging session.

Some embodiments advantageously allow an ultrasound imaging system to provide smart setting and/or preset determination based on determined context parameters of an ultrasound imaging session.

Ultrasound imaging devices may be used to image internal tissue, bones, blood flow, or organs of human or animal bodies in a non-invasive manner. The images can then be displayed. To perform ultrasound imaging, the ultrasound imaging devices transmit an ultrasonic signal into the body and receive a reflected signal from the body part being imaged. Such ultrasound imaging devices include transducers and associated electronics, which may be referred to as transceivers or imagers, and which may be based on photo-acoustic or ultrasonic effects. Such transducers may be used for imaging and may be used in other applications as well. For example, the transducers may be used in medical imaging; flow measurements in arteries and pipes, can form speakers and microphone arrays; can perform lithotripsy; localized tissue heating for therapeutic; and highly intensive focused ultrasound (HIFU) surgery.

Additional aspects and advantages of some embodiments will become readily apparent to those skilled in this art from the instant detailed description, wherein only illustrative embodiments are shown and described. As will be realized, some embodiments are capable of achieving other, different goals, and their several details are capable of modifications in various obvious respects, all without departing from the disclosure. Accordingly, the drawings and description are to be regarded as illustrative in nature, and not as restrictive.

Traditionally, imaging devices such as ultrasound imagers used in medical imaging use piezoelectric (PZT) materials or other piezo ceramic and polymer composites. Such imaging devices may include a housing to house the transducers with the PZT material, as well as other electronics that form and display the image on a display unit. To fabricate the bulk PZT elements or the transducers, a thick piezoelectric material slab may be cut into large rectangular shaped PZT elements. These rectangular-shaped PZT elements may be expensive to build, since the manufacturing process involves precisely cutting generally the rectangular-shaped thick PZT or ceramic material and mounting it on substrates with precise spacing. Further, the impedance of the transducers is much higher than the impedance of the body tissue, which can affect performance.

Still further, such thick bulk PZT elements can require very high voltage pulses, for example 100 volts (V) or more to generate transmission signals. This high drive voltage can sometimes result in high power dissipation since the power dissipation in the transducers is proportional to the square of the drive voltage. This high power dissipation generates heat within the ultrasound imaging device such that cooling arrangements are necessitated. These cooling systems increase the manufacturing costs and weights of the ultrasound imaging devices which makes the ultrasound imaging devices more burdensome to operate.

Some embodiments may be utilized in the context of imaging devices that utilize either piezoelectric micromachined ultrasound transducer (pMUT) or capacitive micromachined ultrasonic transducer (cMUT) technologies, as described in further detail herein.

In general, MUTs, such as both cMUTs and pMUTs, include a diaphragm (a thin membrane attached at its edges, or at some point in the interior of the probe), whereas a “traditional,” bulk PZT element typically consists of a solid piece of material.

Piezoelectric micromachined ultrasound transducers (pMUTs) may be efficiently formed on a substrate leveraging various semiconductor wafer manufacturing operations. Semiconductor wafers may come in 6 inch, 8 inch, and 12 inch sizes and are capable of housing hundreds of transducer arrays. These semiconductor wafers start as a silicon substrate on which various processing operations are performed. An example of such an operation is the formation of SiO2 layers, also known as insulating oxides. Various other operations such as the addition of metal layers to serve as interconnects and bond pads are performed to allow connection to other electronics. Yet another example of a machine operation is the etching of cavities. Compared to the conventional transducers having bulky piezoelectric material, pMUT elements built on semiconductor substrates are less bulky, are cheaper to manufacture, and have simpler and higher performance interconnection between electronics and transducers. As such, they provide greater flexibility in the operational frequency of the ultrasound imaging device using the same, and potential to generate higher quality images. Frequency response may for example be expanded though flexibility of shaping the diaphragm and its active areas with piezo material.

In some embodiments, the ultrasound imaging device includes an application specific integrated circuit (ASIC) that includes transmit drivers, measurement circuitry for received echo signals, and control circuitry to control various operations. The ASIC may be formed on the same or another semiconductor wafer. This ASIC may be placed in close proximity to pMUT or cMUT elements to reduce parasitic losses. As a specific example, the ASIC may be 50 micrometers (μm) or less away from the transducer array. In a broader example, there may be less than 100 μm separation between the 2 wafers or 2 die, where each wafer includes many die, and a die includes a transducer array in the transducer wafer and an ASIC array in the ASIC wafer. The array may have up to 10,000 or more individual elements. In some embodiments, the ASIC has matching dimensions relative to the pMUT or cMUT array and allows the devices to be stacked for wafer-to-wafer interconnection or transducer die on ASIC wafer or transducer die to ASIC die interconnection. Alternatively, the transducer can also be developed on top of the ASIC wafer using low temperature piezo material sputtering and other low temperature processing compatible with ASIC processing.

Wherever the ASIC and the transducer interconnect, according to one embodiment, the two may have similar footprints. More specifically, according to the latter embodiment, a footprint of the ASIC may be an integer multiple or divisor of the MUT footprint.

Regardless of whether the ultrasound imaging device is based on pMUT or cMUT, an imaging device according to some embodiments may include a number of transmit channels and a number of receive channels. Transmit channels are to drive the transducer elements with a voltage pulse at a frequencies the elements are responsive to. This may cause an ultrasonic waveform to be emitted from the elements, which waveform is to be directed towards an object to be imaged (target object), such as toward an organ or other tissue in a body. In some examples, the ultrasound imaging device with the array of transducer elements may make mechanical contact with the body using a gel in between the ultrasound imaging device and the body. The ultrasonic waveform travels towards the object or target, i.e., an organ, and a portion of the waveform is reflected back to the transducer elements in the form of received/reflected ultrasonic energy where the received ultrasonic energy may be converted to an electrical energy within the ultrasound imaging device. The received ultrasonic energy may be processed by a number of receive channels to convert the received ultrasonic energy to signals, and the signals may be processed by other circuitry to develop an image of the object for display based on the signals.

An embodiment of an ultrasound imaging device includes a transducer array, and control circuitry including, for example, an application-specific integrated circuit (ASIC), and transmit and receive beamforming circuitry, and optionally additional control electronics.

In an embodiment, an imaging device may include a handheld casing or handheld housing where transducers and associated electronic circuitries, such as a control circuitry and optionally a computing device are housed. The ultrasound imaging device may also contain a battery to power the electronic circuitries.

Thus, some embodiments pertain to a portable imaging device utilizing either pMUT elements or cMUT elements in a 2D array. In some embodiments, such an array of transducer elements is coupled to an application specific integrated circuit (ASIC) of the ultrasound imaging device.

An ultrasound exam (or “scanning operation” or “ultrasonic imaging session” or “imaging session”) may be associated with one of a number of possible ultrasound presets. An individual ultrasound preset may be associated with one or more ultrasound settings. Each setting can be changed or controlled independently or in dependence of other settings within a preset to improve ultrasound imaging.

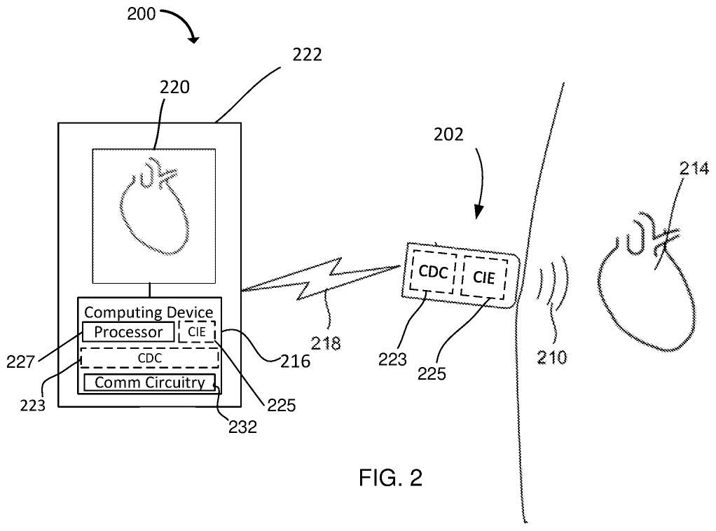

Some examples of ultrasound settings include:
a) Gain: the gain setting may control the brightness of the ultrasonic image shown on a display, such as display 220 of FIG. 2. Increasing the gain can make the image brighter, while decreasing the gain can make the image darker.
b) Depth: the depth setting controls how deep the ultrasound waves penetrate into the body. Increasing the depth can allow for imaging deeper structures, while decreasing the depth can provide more detailed imaging of shallow structures.
c) Frequency: the frequency setting controls the frequency of the ultrasound waves per transducer element. Higher frequency ultrasound waves provide better image resolution for superficial structures, while lower frequency ultrasound waves can penetrate deeper into the body for imaging deeper structures.
d) Time gain compensation (TGC): TGC settings adjust the gain of the ultrasound signal at different depths. This helps to ensure that the image is uniformly bright at all depths.
e) Dynamic range: the dynamic range setting adjusts the range of brightness levels that can be displayed in the image. Increasing the dynamic range can improve the visibility of subtle differences in tissue texture or echogenicity.
f) Focus: the focus setting adjusts where the ultrasound waves converge in the body. This can help to improve image resolution and clarity at a specific depth.
g) Harmonics: the harmonics setting allows the probe to emit and receive sound waves at higher frequencies than a fundamental frequency of the probe. Harmonics are multiples of the fundamental frequency, and they can be used to provide higher resolution images in certain medical imaging applications. In medical ultrasonography, the fundamental frequency of a probe may be in the range between about 2 to about 10 MHz or higher, such as possibly up to 18 MHz by way of example.
h) Mode:
   1. Doppler: the Doppler mode of an ultrasound imaging probe refers to a mode that allows the probe to detect and measure the velocity and direction of blood flow in the body. This is done by using the Doppler effect, which is the change in frequency of sound waves that occurs when they are reflected off moving objects. In medical ultrasonography, the Doppler setting is often used to assess blood flow in arteries and veins. The probe emits sound waves at a specific frequency that is reflected off the moving red blood cells in the vessel. The returning sound waves are then analyzed to determine the velocity and direction of the blood flow.
   2. B-mode: the B mode produces a two-dimensional grayscale image of the body. B-mode may use a single transducer to emit sound waves and then receive the reflected echoes to create an image.
   3. M-mode: This mode is used to visualize motion within a specific region of the body over time. It produces a one-dimensional image in which the x-axis represents time, and the y-axis represents the position of the moving structure.
   4. 3D/4D mode: This mode is used to create three-dimensional images of the body, allowing for better visualization and assessment of complex anatomical structures. 4D mode adds the element of time to the 3D image, producing a real-time view of the body in motion.
i) Focal zone: Focal zone pertains to the region in the body where the ultrasonic beam is focused to achieve the best image quality. The focal zone is determined by a transducer element's aperture and the frequency of the emitted sound waves. Typically, an ultrasound imaging probe has multiple focal zones that can be adjusted by the user.
j) Persistence: Persistence is a feature that enables the display of previous ultrasound frames on the screen in a semi-transparent manner. This can help reduce speckle noise and enhance the visualization of structures that are moving slowly.
k) Automatic gain control (AGC): This is a feature that adjusts the gain of the ultrasound signal to maintain a consistent brightness level across the entire image. AGC can be set to “auto” or “manual” mode, and some probes have additional features such as tissue equalization, which can further enhance image quality.
l) Spatial compounding: Spatial compounding is a technique that involves combining multiple ultrasound images from different angles to create a single composite image with reduced noise and improved contrast resolution.
m) Frequency compounding: Frequency compounding is a technique that involves using different frequencies of ultrasound waves to create a single image. This can improve image quality by reducing the effects of attenuation and scattering of sound waves in tissues.
n) Sine functions: Sine functions are used in ultrasound imaging to modulate the amplitude, frequency, or phase of the emitted sound waves. This can help to reduce artifacts and improve the quality of the resulting image.
o) Line density: Line density refers to the number of scan lines used to create an image. Higher line density can improve image resolution but also increases the amount of data that needs to be processed.
p) Tint maps or tint maps configuration: Tint maps are color-coded maps used to display different types of information in an ultrasound image. For example, different shades of blue and red can be used to indicate the direction and velocity of blood flow in a vessel. Tint maps can help to make it easier for the user to interpret the information presented in the image.

Other examples of settings include: measurements, annotations, settings for tissue border delineation, to name a few.

A given preset may be associated with a cluster (or set) of ultrasound settings. Examples of presets for ultrasound imaging include:
i. Abdominal: This preset may be optimized for imaging the abdominal region, including the liver, pancreas, and kidneys. It typically uses a low to medium frequency and a wide dynamic range to capture details in the deep structures of the abdomen.
ii. Renal: This preset may be specifically optimized for imaging the kidneys and detecting any abnormalities such as cysts, tumors, or obstructions. It typically uses a medium frequency and has specialized settings for measuring renal blood flow and identifying renal stones.
iii. Obstetrics: This preset may be optimized for imaging the fetus during pregnancy. It typically uses a high frequency and has specialized settings for measuring fetal biometry and assessing fetal growth and development.
iv. Cardiac: This preset may be optimized for imaging the heart and cardiovascular system. It typically uses a high frequency and specialized settings for measuring blood flow, cardiac function, and detecting abnormalities such as valve stenosis or regurgitation.
v. Musculoskeletal: This preset may be optimized for imaging the musculoskeletal system, including bones, joints, and soft tissue. It typically uses a high frequency and has specialized settings for detecting fractures, tendon tears, and other soft tissue injuries.
vi. Breast: This preset may be optimized for imaging the breast and detecting abnormalities such as tumors or cysts. It typically uses a high frequency and has specialized settings for detecting microcalcifications and characterizing breast lesions.
vii. Thyroid: This preset may be optimized for imaging the thyroid gland and detecting abnormalities such as nodules or cysts. It typically uses a high frequency and has specialized settings for measuring thyroid volume and blood flow.

The above are just a few examples of presets for medical ultrasound imaging probes. Different manufacturers and models of probes may have different presets or customizable settings based on the specific imaging needs of the user.

In the following description, for purposes of explanation, specific details are set forth in order to provide an understanding of the disclosure. It will be apparent, however, to one skilled in the art that the disclosure may be practiced without these details. Furthermore, one skilled in the art will recognize that examples of the present disclosure, described below, may be implemented in a variety of ways, such as a process, one or more processors (processing circuitry) of a control circuitry, one or more processors (or processing circuitry) of a computing device, a system, a device, or a method on a tangible computer-readable medium.

One skilled in the art shall recognize: (1) that certain fabrication operations may optionally be performed; (2) that operations may not be limited to the specific order set forth herein; and (3) that certain operations may be performed in different orders, including being done contemporaneously, and (4) operations may involve using Artificial Intelligence.

Elements/components shown in diagrams are illustrative of exemplary embodiments and are meant to avoid obscuring the disclosure. Reference in the specification to “one example,” “preferred example,” “an example,” “examples,” “an embodiment,” “some embodiments,” or “embodiments” means that a particular feature, structure, characteristic, or function described in connection with the example is included in at least one example of the disclosure and may be in more than one example. The appearances of the phrases “in one example,” “in an example,” “in examples,” “in an embodiment,” “in some embodiments,” or “in embodiments” in various places in the specification are not necessarily all referring to the same example or examples. The terms “include,” “including,” “comprise,” and “comprising” shall be understood to be open terms and any lists that follow are examples and not meant to be limited to the listed items. Any headings used herein are for organizational purposes only and shall not be used to limit the scope of the description or the claims. Furthermore, the use of certain terms in various places in the specification is for illustration and should not be construed as limiting.

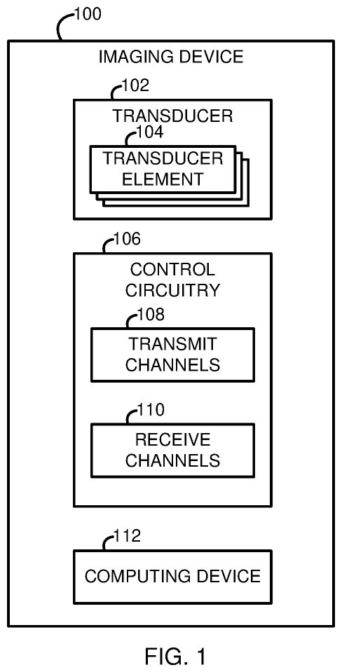

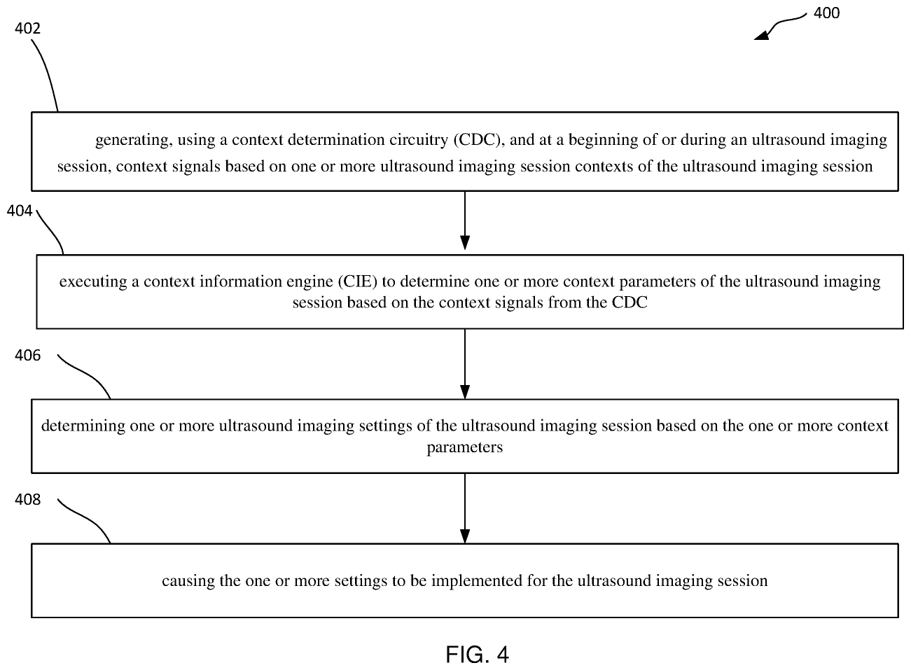

Reference will be made to FIGS. 1-3, which show devices and circuitries that may be used to implement some embodiments as described herein. Reference will further be made to FIGS. 4A and 4B which show an ultrasound imaging device according to the state of the art. Reference will thereafter be made to FIGS. 5A and 5B, which show an ultrasound imaging device according to one embodiment being held in two different manners.

Turning now to the figures, FIG. 1 is a block diagram of an imaging device 100 with a controller or control circuitry 106 controlling selectively alterable channels (108, 110) and having imaging computations performed on a computing device 112 according to principles described herein. As described above, the ultrasound imaging device 100 may be used to generate an image of internal tissue, bones, blood flow, or organs of human or animal bodies. Accordingly, the ultrasound imaging device 100 may transmit a signal into the body and receive a reflected signal from the body part being imaged. Such imaging devices may include either pMUT or cMUT, which may be referred to as transducers or imagers, which may be based on photo-acoustic or ultrasonic effects. The ultrasound imaging device 100 may be used to image other objects as well. For example, the ultrasound imaging device may be used in medical imaging; flow measurements in pipes, speaker, and microphone arrays; lithotripsy; localized tissue heating for therapeutic; and highly intensive focused ultrasound (HIFU) surgery.

In addition to use with human patients, the ultrasound imaging device 100 may be used to acquire an image of internal organs of an animal as well. Moreover, in addition to imaging internal organs, the ultrasound imaging device 100 may also be used to determine direction and velocity of blood flow in arteries and veins as in Doppler mode imaging and may also be used to measure tissue stiffness.

The ultrasound imaging device 100 may be used to perform different types of imaging. For example, the ultrasound imaging device 100 may be used to perform one-dimensional imaging, also known as A-Scan, two-dimensional imaging, also known as B scan, three-dimensional imaging, also known as C scan, and Doppler imaging (that is, the use of Doppler ultrasound to determine movement, such as fluid flow within a vessel). The ultrasound imaging device 100 may be switched to different imaging modes, including without limitation linear mode and sector mode, and electronically configured under program control.

To facilitate such imaging, the ultrasound imaging device 100 includes one or more ultrasound transducers 102, each transducer 102 including an array of ultrasound transducer elements 104. Each ultrasound transducer element 104 may be embodied as any suitable transducer element, such as a pMUT or cMUT element. The transducer elements 104 operate to 1) generate the ultrasonic pressure waves that are to pass through the body or other mass and 2) receive reflected waves (received ultrasonic energy) off the object within the body, or other mass, to be imaged. In some examples, the ultrasound imaging device 100 may be configured to simultaneously transmit and receive ultrasonic waveforms or ultrasonic pressure waves (pressure waves in short). For example, control circuitry 106 may be configured to control certain transducer elements 104 to send pressure waves toward the target object being imaged while other transducer elements 104, at the same time, receive the pressure waves/ultrasonic energy reflected from the target object, and generate electrical charges based on the same in response to the received waves/received ultrasonic energy/received energy.

In some examples, each transducer element 104 may be configured to transmit or receive signals at a certain frequency and bandwidth associated with a center frequency, as well as, optionally, at additional center frequencies and bandwidths. Such multi-frequency transducer elements 104 may be referred to as multi-modal elements 104 and can expand the bandwidth of the ultrasound imaging device 100. The transducer element 104 may be able to emit or receive signals at any suitable center frequency, such as about 0.1 to about 100 megahertz.

To generate the pressure waves, the ultrasound imaging device 100 may include a number of transmit (Tx) channels 108 and a number of receive (Rx) channels 110. The transmit channels 108 may include a number of components that drive the transducer 102, i.e., the array of transducer elements 104, with a voltage pulse at a frequency that they are responsive to. This may cause an ultrasonic waveform to be emitted from the transducer elements 104 towards an object to be imaged.

According to some embodiments, an ultrasonic waveform may include one or more ultrasonic pressure waves transmitted from one or more corresponding transducer elements of the ultrasound imaging device substantially simultaneously.

The ultrasonic waveform travels towards the object to be imaged (target) and a portion of the waveform is reflected back to the transducer 102, which converts it to an electrical energy through a piezoelectric effect. The receive channels 110 collect electrical energy thus obtained, and process it, and send it for example to the computing device 112, which develops or generates an image that may be displayed.

In some examples, the number of transmit channels 108 and receive channels 110 in the ultrasound imaging device 100 may remain constant, although the coupling of respective transducer elements to the transmit channels 108 and receive channels 110 may vary, for example based on coupling schemes dictated by the control circuitry. A coupling of the transmit and receive channels to the transducer elements may be, in one embodiment, controlled by control circuitry 106. In some examples, for example as shown in FIG. 1, the control circuitry may include the transmit channels 108 and the receive channels 110. For example, the transducer elements 104 of a transducer 102 may be formed into a two-dimensional spatial array with N columns and M rows. In a specific example, the two-dimensional array of transducer elements 104 may have 128 columns and 32 rows. In this example, the ultrasound imaging device 100 may have up to 128 transmit channels 108 and up to 128 receive channels 110. In this example, each transmit channel 108 and receive channel 110 may be coupled to multiple or single pixels 104. For example, depending on the imaging mode (for example, whether a linear mode where a number of transducers transmit ultrasound waves in a same spatial direction, or a sector mode, where a number of transducers transmit ultrasound waves in different spatial directions), each column of transducer elements 104 may be coupled to a single transmit channel 108 and a single receive channel (110). In this example, the transmit channel 108 and receive channel 110 may receive composite signals, which composite signals combine signals received at each transducer element 104 within the respective column. In another example, i.e., during a different imaging mode, each transducer element 104 may be coupled to its dedicated transmit channel 108 and its dedicated receive channel 110. In some embodiments, a transducer element 104 may be coupled to both a transmit channel 108 and a receive channel 110. For example, a transducer element 104 may be adapted to create and transmit an ultrasound pulse and then detect the echo of that pulse in the form of converting the reflected ultrasonic energy into electrical energy.

The control circuitry 106 may be embodied as any circuit or circuits configured to perform the functions described herein. For example, the control circuitry 106 may be embodied as or otherwise include an application specific integrated circuit (ASIC), a field programmable gate array (FPGA), a system-on-a-chip, a processor and memory, a voltage source, a current source, one or more amplifiers, one or more digital-to-analog converters, one or more analog-to-digital converters, etc.

The illustrative computing device 112 may be embodied as any suitable computing device including any suitable components, such as one or more processors (i.e., one or more processing circuitries), one or more memory circuitries, one or more communication circuitries, one or more batteries, one or more displays, etc. In one embodiment, the computing device 112 may be integrated with the control circuitry 106, transducers 102, etc., into a single microelectronic package or single chip, or a single system on a chip (SoC), or a single ultrasound imaging device housing as suggested for example in the embodiment of FIG. 1. In other embodiments, some or all of the computing devices may be in a separate microelectronic package from the control circuitry, or in a separate device distinct from the ultrasound imaging device such as an ultrasound imaging probe, as suggested for example in the embodiment of in FIG. 2 as will be described in further detail below.

Each transducer element may have any suitable shape such as, square, rectangle, ellipse, or circle. The transducer elements may be arranged in a two dimensional array arranged in orthogonal directions, such as in N columns and M rows as noted herein or may be arranged in an asymmetric (or staggered) rectilinear array.

Transducer elements 104 may have associated transmit driver circuits of associated transmit channels, and low noise amplifiers of associated receive channels. Thus, a transmit channel may include transmit drivers, and a receive channel may include one or more low noise amplifiers. For example, although not explicitly shown, the transmit and receive channels may each include multiplexing and address control circuitry to enable specific transducer elements and sets of transducer elements to be activated, deactivated or put in low power mode. It is understood that transducers may be arranged in patterns other than orthogonal rows and columns, such as in a circular fashion, or in other patterns based on the ranges of ultrasonic waveforms to be generated therefrom.

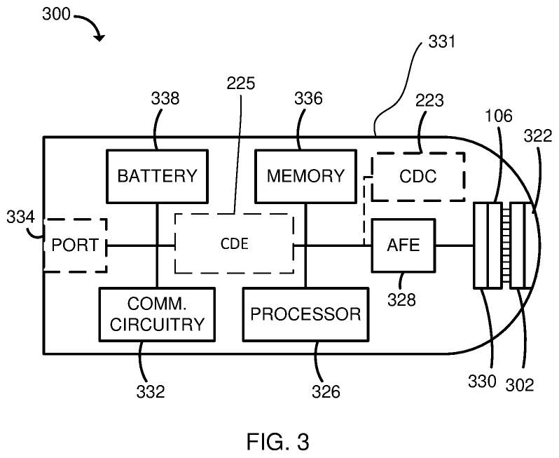

FIG. 2 is a diagram of an imaging environment including an imaging network 200 with selectively configurable characteristics, according to an embodiment. The imaging system of FIG. 2 may include two ultrasound imaging systems, a first ultrasound imaging system in the form of an ultrasound imaging probe 202 (which may be similar to ultrasound imaging probe 100 of FIG. 1, or 300 described below in the context of FIG. 3) and a second ultrasound imaging system in the form of a computing system 222 which includes a computing device 216 and a display 220 coupled to the computing device, as will be described in further detail below.

As depicted in FIG. 2, the computing device 216 may, according to one embodiment, and unlike the embodiment of FIG. 1, be physically separate from the ultrasound imaging probe 202. For example, the computing device 216 and display 220 may be disposed within a separate device (in this context, the shown computing system 222, physically separate from imaging probe 202 during operation (e.g., during an imaging session) as compared with the components of the ultrasound imaging probe 202. The computing system 222 may include a mobile device, such as cell phone or tablet, or a stationary computing device, which can display images to a user. In another example, as shown in FIG. 1 for example, the display device, the computing device, and associated display, may be part of the ultrasound imaging probe 202 (now shown). That is, the ultrasound imaging probe 202, computing device 216, and display 220 may be disposed within a single housing.

In one example, the computing system, such as computing system 222 of FIG. 2, may include a display 220 and a computing device 216 coupled to the display 220. The computing device may include a processor (or processing circuitry) 227, and a communication circuitry 232.

One or both of the computing device 216 or the probe 202 may implement a context information engine (CIE) 225, such as in the form of software, and a context determination circuitry (CDC) 223, as will be explained in further detail below. The CIE and CDC are shown in broken lines in FIG. 2 to suggest that they could be implemented in one or more of the computing device 216 or probe 202.

A “computing device” as referred to herein may, in some embodiments, be configured to generate signals to at least one of cause an image of the object to be displayed on a display, or cause information regarding the image to be communicated to a user.

A “computing device,” as referred to herein may, in some embodiments, be configured to use context information regarding one or more context parameters of the ultrasound imaging probe (hereinafter “context parameter signals”), and to process those context parameter signals to cause generation of control signals to change one or more settings of an imaging session by the ultrasound imaging device.

An “ultrasound imaging system” as referred to herein may refer to an ultrasound imaging probe, or to a computing system that is paired or is to be paired with but distinct from an ultrasound imaging probe that that is to receive signals from the ultrasound imaging probe.

A “context parameter of an ultrasound imaging system” or “ultrasound imaging system context parameter” or “context parameter” as used herein refers to information regarding a circumstance in which the ultrasound imaging system is used or to be used to execute an ultrasound imaging session. A context parameter may include a dynamic context parameter, that is, information about a circumstance of the ultrasound imaging system that is subject to change.

Some embodiments include a scenario where a first ultrasound imaging system's CDC generates a context signal, and a second ultrasound imaging system's processor determines the settings for the imaging session, where the first and second ultrasound imaging systems are paired to one another.

Examples of a “context parameter” may include any of the following information:
(1) information on the patient to be or being imaged (“patient information,” e.g., identity of patient, history of prior patient imaging sessions, history of prior patient diagnoses, bodily characteristics of patient (e.g., body mass index (BMI)), patient gender);
(2) information regarding the clinician to perform or performing the imaging session (“clinician information,” e.g., clinician name, clinician specialty, history of imaging session settings for the clinician, history of imaging session types for the clinician);
(3) imaging session location information (e.g., geographic and/or indoor location);
(4) time information (including date (day, month and/or year) of performing the imaging session;
(5) information on ambient light at a location where images of the imaging session are to be or are being displayed (e.g., sunlight, artificial light);
(6) information on the type(s) of display device(s) to be or being used for the imaging session;
(7) auditory or sound information at a location where an imaging session is to take place or is taking place (e.g., information relating to communication between a clinician and patient, which can be processed at the CIE using word recognition (e.g., “the gel on your abdomen is going to feel cold” may be used by the processing circuitry to determine abdomen presets); or
(8) visual information at the location where an imaging session is to take place or is taking place (e.g., by way of a camera, information relating to a clinician or a patient, which can be processed at the CIE using image recognition to identify a clinician or patient), to name a few examples.

A “clinician” as referred to herein may refer to at least one of:
A. an operator of an ultrasound imaging probe (the operator holding the probe and navigating it over a body during an ultrasound imaging session); or
B. an operator of a computing system having a display device that shows images corresponding to an ultrasound imaging session being performed through the use of an ultrasound imaging probe.

Where the ultrasound imaging probe and the computing system are in a same location (either because they are integrated in a same device, or because they are distinct from one another but in a same location nevertheless), it is possible that the operator in A. above and the operator in B. above may be the same.

A “patient” as referred to herein may refer to a body to be or being subject to an ultrasound imaging session, such as the body or body part of a human or other animal, dead or alive.

For example, an operator may be using a probe during an imaging session at location X, and a smartphone or tablet nearby at a same location X may be showing the same operator images relating to the imaging session. Alternatively or simultaneously, an operator may be using a probe during an imaging session at location X, and a different operator may be seeing and assessing images relating to the imaging session on a display device at location Y different from location X.

An “operator” as referred to herein may be a person, or a machine programmed to perform as a sonographer.

A “circuitry” as referred to herein may refer to a single circuit, or to multiple circuits. The multiple circuits may be implemented in a single physical device, or in a distributed manner across multiple physical devices.

As depicted, the imaging system includes the ultrasound imaging probe 202 that is configured to generate and transmit, via the transmit channels (FIG. 1, 108), pressure waves 210 toward an object, such as a heart 214, in a transmit mode/process. The internal organ, or other object to be imaged, may reflect a portion of the pressure waves 210 toward the ultrasound imaging probe 202 which may receive, via a transducer (such as transducer 102 of FIG. 1), receive channels (FIG. 1, 110), control circuitry (FIG. 1, 106), the reflected pressure waves. The transducer may generate an electrical signal based on the received ultrasonic energy in a receive mode/process. A transmit mode or receive mode may be applicable in the context of imaging devices that may be configured to either transmit or receive, but at different times. However, as noted previously, some imaging devices according to embodiments may be adapted to be in both a transmit mode and a receive mode simultaneously. The system also includes a computing device 216 that is to communicate with the ultrasound imaging probe 202 through a communication channel, such as a wireless communication channel 218 as shown, although embodiments also encompass within their scope wired communication between a computing system and imaging device. The ultrasound imaging probe 202 may communicate signals to the computing device 216 which may have a processor (or processing circuitry) 227 to process image-related data from the ultrasound probe 202 and to use the received signals to complete formation of an image of the object on display. A display 220 of the computing system 222 may then display images of the object using the signals from the computing device.

An imaging device according to some embodiments may include a portable device, and/or a handheld device that is adapted to communicate signals through a communication channel, either wirelessly (using a wireless communication protocol, such as an IEEE 802.11 or Wi-Fi protocol, a Bluetooth protocol, including Bluetooth Low Energy, a mmWave communication protocol, or any other wireless communication protocol as would be within the knowledge of a skilled person) or via a wired connection such as a cable (such as USB2, USB 3, USB 3.1, and USB-C) or such as interconnects on a microelectronic device, with the computing device. In the case of a tethered or wired connection, the ultrasound imaging device may include a port for receiving a cable connection of a cable that is to communicate with the computing device. In the case of a wireless connection, the ultrasound imaging probe 202 may include a wireless transceiver to communicate with the computing device 216.

It should be appreciated that, in various embodiments, different aspects of the disclosure may be performed in different components. For example, in one embodiment, the ultrasound imaging device may include circuitry (such as the channels) to cause ultrasound waveforms to be sent and received through its transducers, while the computing device may be adapted to control such circuitry to the generate ultrasound waveforms at the transducer elements of the ultrasound imaging device using voltage signals, and further a processing of the received ultrasonic energy.

FIG. 3 represents a view of an imaging device according to some embodiments, as will be described in further detail below.

As seen in FIG. 3, the ultrasound imaging probe 300 may include a handheld casing or housing 331 where transducers 302 and associated electronics are housed. The ultrasound imaging device may also contain a battery 338 to power the electronics. FIG. 3 thus shows an embodiment of a portable imaging device capable of 2D and 3D imaging using pMUTs in a 2D array, optionally built on a silicon wafer. Such an array coupled to an application specific integrated circuit (ASIC) 106 with electronic configuration of certain parameters, enables a higher quality of image processing at a lower cost than has been previously possible. Further by certain data, for example relating to the number of channels used, power consumption.

FIG. 3 is a schematic diagram of an imaging device such as an ultrasound imaging probe 300 with selectively adjustable features, according to some embodiments. The ultrasound imaging probe 300 may be similar to imaging device 100 of FIG. 1, or to imaging probe 202 of FIG. 2, by way of example only. FIG. 3 depicts transducer(s) 302 of the ultrasound imaging probe 300. As described above, the transducer(s) 302 may include arrays of transducer elements (FIG. 1, 104) that are adapted to transmit and receive pressure waves (FIG. 2, 210). In some examples, the ultrasound imaging probe 300 may include a coating layer 322 that serves as an impedance matching interface between the transducers 302 and the human body, or other mass or tissue through which the pressure waves (FIG. 2, 210) are transmitted. In some cases, the coating layer 322 may serve as a lens when designed with the curvature consistent with focal length desired.

The ultrasound imaging probe 300 housing 331 may be embodied in any suitable form factor. In some embodiments, part of the ultrasound imaging probe 300 that includes the transducers 302 may extend outward from the rest of the ultrasound imaging probe 300. The ultrasound imaging probe 300 may be embodied as any suitable ultrasonic medical probe, such as a convex array probe, a micro-convex array probe, a linear array probe, an endovaginal probe, endorectal probe, a surgical probe, an intraoperative probe, etc.

In some embodiments, the user may apply gel on the skin of a living body before a direct contact with the coating layer 322 so that the impedance matching at the interface between the coating layer 322 and the human body may be improved. Impedance matching reduces the loss of the pressure waves (FIG. 2, 210) at the interface and the loss of the reflected wave travelling toward the ultrasound imaging probe 300 at the interface.

In some examples, the coating layer 322 may be a flat layer to maximize transmission of acoustic signals from the transducer(s) 102 to the body and vice versa. The thickness of the coating layer 322 may be a quarter wavelength of the pressure wave (FIG. 2, 210) to be generated at the transducer(s) 102.

The ultrasound imaging probe 300 also includes a control circuitry 106, such as one or more processors, optionally in the form of an application-specific integrated circuit (ASIC chip or ASIC), for controlling the transducers 102. The control circuitry 106 may be coupled to the transducers 102, such as by way of bumps.

An ultrasound imaging system according to some embodiments may implement a context information engine (CIE) 225 (as will be described further below) and a context determination circuitry (CDC) 223 as will be described further below.

The ultrasound imaging probe may also include one or more processors (or processing circuitries) 326 for controlling the components of the ultrasound imaging probe 300. One or more processors 326 may be configured to, in addition to control circuitry 106, at least one of control an activation of transducer elements, process signals based on reflected ultrasonic waveforms from the transducer elements or generate signals to cause generation of an image of an object being imaged by one or more processors of a computing device, such as computing device 112 of FIG. 1 or 216 of FIG. 2. One or more processors 326 may further be adapted to perform other processing functions associated with the ultrasound imaging probe.

The one or more processors 326 may be embodied as any type of processors 326. For example, the one or more processors 326 may be embodied as a single or multi-core processor(s), a single or multi-socket processor, a digital signal processor, a graphics processor, a neural network compute engine, an image processor, a microcontroller, a field programmable gate array (FPGA), or other processor or processing/controlling circuit.

The ultrasound imaging probe 300 may also include circuitry 328, such as Analog Front End (AFE), for processing/conditioning signals. The analog front end 328 may be embodied as any circuit or circuits configured to interface with the control circuitry 106 and other components of the ultrasound imaging probe, such as the processing circuitry 326. For example, the analog front end 328 may include, e.g., one or more digital-to-analog converters, one or more analog-to-digital converters, one or more amplifiers, etc.

The ultrasound imaging probe may include a communication circuitry 332 for communicating data, including control signals, with an external device, such as the computing device (FIG. 2, 216), through for example an input/output (I/O) circuitry 334 of the probe 300. The communication circuitry 332 may, for example, include signal processing circuitry for signals communicated through the I/O circuitry. For example, the communication circuitry may include one or more of a baseband processor, a modem, digital signal processing (DSP) circuitry, an analog-to-digital converter (ADC), or an equalizer circuitry.

For example, the I/O circuitry 334 may include one or more ports for wired communication with the probe 300, or a wireless transceiver circuitry 335 for wireless communication with the probe 300. The wireless transceiver circuitry 335 may, for example, include one or more transmit (TX) circuitries to transmit signals from the ultrasound imaging probe 300, and one or more receive (RX) circuitries to receive signals into the ultrasound imaging probe 300. The TX and RX circuitries may include TX and RX ports within port 334, which may, for example, correspond to a USB port. The TX and RX circuitries may include TX chains and RX chains of one or more wireless transceivers. A TX chain or an RX chain may include, for example, one or more antennas, amplifiers, filters, and/or mixers.

The ultrasound imaging probe 300 may include memory circuitry 336 for storing data. The memory circuitry 336 may be embodied as any type of volatile or non-volatile memory or data storage capable of performing the functions described herein. In operation, the memory circuitry 336 may store various data and software used during operation of the ultrasound imaging probe 300 such as operating systems, applications, programs, libraries, and drivers.

In some examples, the ultrasound imaging probe 300 may include a battery 338 for providing electrical power to the components of the ultrasound imaging probe 300. The battery 338 may also include battery charging circuits which may be wireless or wired charging circuits (not shown). The ultrasound imaging probe may include a gauge that indicates a battery charge consumed and is used to configure the ultrasound imaging probe to optimize power management for improved battery life. Additionally or alternatively, in some embodiments, the ultrasound imaging probe may be powered by an external power source, such as by plugging the ultrasound imaging probe into a wall outlet.

An ultrasound imaging system such as either the ultrasound imaging probe 100/300 (FIGS. 2/3) and/or the computing device 216 (FIG. 2) may include a context information engine (CIE) 235 and a context determination circuitry (CDC) 223.

A CIE according to embodiments is to determine context information for the ultrasound imaging system. Context information includes a set of one or more context parameters that may be used by the processing circuitry to determine one or more settings of the ultrasound imaging system. Examples of context parameters are provided above, such as at items (1)-(8). Examples of settings are provided above, such as at items a) through p) and i. through vii.

A CDC according to embodiments is to generate context signals for example by measuring contexts (e.g., where the CDC includes measurement circuitries in the form of sensors (e.g., microphones, light sensors, cameras) or clocks), otherwise capturing contexts through access to a memory to read data on the history of a patient, clinician and/or prior imaging sessions, or by decoding context signals received from another system (e.g., where the CDC decodes information sent from another ultrasound imaging system that indicates the type of display for an imaging session).

Thus, the CIE may determine the context information by accessing context signals at least one of generated at (e.g., by the CDC) or received at (e.g., as communicated by another device to a CDC) the ultrasound imaging system. The context signals may be generated at the ultrasound imaging system by a CDC 223, which may be in the form of one or more measurements circuitries (e.g., sensors or clocks). The context signals may be received by the ultrasound imaging system through a communication circuitry thereof, such as communication circuitry 223 of computing device 216 or communication circuitry 334 of probe 300.

A measurement circuitry according to some embodiments may correspond to a sensor circuitry to measure a physical phenomenon (e.g., to sense light, sound, radio waves (e.g., in the case of a GPS sensor). The measurement circuitry according to an embodiment may include one or more clocks. The measurement circuitry may generate the context signals based on the sensed physical phenomenon, and send these context signals to the CIE 22. The ultrasound imaging system may include multiple measurements circuitries to sense multiple physical phenomena and to generate distinct sets of corresponding context signals, and the CIE 225 may be configured to generate context information based on one or more context signals.

For example, context signals may correspond to time of day measured by a clock, and location measured by a GPS. A CIE 225 may use the time context signals and the location context signals to infer that the context parameter corresponds to clinician information, specifically to information that the clinician is an echocardiographer. The latter determination by the CIE 225 may be based, for example, on historical information used by the CIE 225 (and accessed from a memory) and corresponding to the fact that, for the last X ultrasound imaging sessions, the given time information and location information have corresponded to a clinician who is an echocardiographer. The CIE may then send the clinician information to the processing circuitry 227/326, which may determine the preset for the imaging session to correspond to a cardiac preset.

According to some embodiments, at least one of the CIE or the CDC of an ultrasound imaging system may be executed by/part of the processing circuitry of the ultrasound imaging system. Alternatively, the CIE and/or the CDC may be executed by/part of a circuitry distinct from the processing circuitry.

For example, the CIE be configured to process visual signals from a CDC that is in the form of one or more visual information sensors. The one or more visual information sensors may be on the probe or on the computing system or both, and may send visual signals to the CIE 225. The CIE may for example correspond to an image recognition software that may recognize facial features or visual information on a badge or other visual information regarding the patient and/or the clinician.

According to an embodiment, the CIE may determine one or more context parameters (“context information,” e.g., patient information, clinician information, location information, time information, ambient light information, type of display information) from the visual signals, and may provide the determined context information to a processing circuitry 227/326 (e.g., at least one of processing circuitry 227 of computing device 216, or processing circuitry 326 of probe 300). The processing circuitry 227/326 may then cause a change in one or more settings of the ultrasound imaging session based on the determined context information.

A visual information sensor according to some embodiments may for example include a camera or a thermal imaging sensor, to name a few.

For example, the CIE be configured to process sound signals from a CDC that is in the form of one or more sound sensors. The one or more sound sensors may be on the probe or on the computing system or both, and may send sound signals to the CIE 225. The CIE may for example correspond to a sound recognition software that may recognize sound information (e.g., spoken words, voice or sound patterns associated with a given person) to identify the patient and/or the clinician.

A sound sensor according to some embodiments may for example include a microphone, a piezoelectric sensor, an accelerometer, a fiber optic sensor or a laser doppler vibrometer, to name a few.

For example, the CIE be configured to process location signals from a CDC that is in the form of one or more location sensors in order to identify location information regarding an imaging session to be performed or being performed. The one or more location sensors may be on the probe or on the computing system or both, and may send location signals to the CIE 225. The CIE may for example correspond to a location recognition software that may recognize location information (e.g., information regarding a location as identified by a location sensor, such as, for example, a recognition that location signals corresponds to a specialist's office (e.g., cardiology, endocrinology, rheumatology, ophthalmology, orthopedics, gynecology, urology, gastroenterology, endocrinology, neurology), a recognition that one is outdoors, a recognition that one is not at a medical facility, etc.)

According to an embodiment, the CIE may determine one or more context parameters (“context information,” e.g., time information, patient information, clinician information, ambient light information, type of display information, location information) from the location signals, and may provide the determined context information to a processing circuitry 227/326 (e.g., at least one of processing circuitry 227 of computing device 216, or processing circuitry 326 of probe 300). The processing circuitry 227/326 may then cause a change in one or more settings of the ultrasound imaging session based on the determined context information.

A location sensor according to some embodiments may for example include a GPS (Global Positioning System) sensor, a WiFi positioning sensor (e.g., a sensor that uses WiFi signals to locate objects or people within a certain range of WiFi access points), a Bluetooth Low Energy (BLE) sensor (e.g., a sensor that is to use Bluetooth signals to locate nearby objects or people within a certain range, or a RFID (Radio Frequency Identification) sensor (e.g., a sensor that is to use radio waves to identify and locate objects that have RFID tags attached to them), to name a few.

For example, the CIE be configured to process time signals from a CDC that is in the form of one or more clocks in order to identify time information (time of day (including night), date, day of week, month of year, time range) regarding an imaging session to be performed or being performed. The one or more clocks may be on the probe or on the computing system or both, and may send time signals to the CIE 225. The CIE may for example be configured to determine one or more context parameters (“context information,” e.g., time information, patient information, clinician information, ambient light information, type of display information, location information) from the time information, and may provide the determined context information to a processing circuitry 227/326 (e.g., at least one of processing circuitry 227 of computing device 216, or processing circuitry 326 of probe 300). The processing circuitry 227/326 may then cause a change in one or more settings of the ultrasound imaging session based on the determined context information.

According to an embodiment, the CIE may determine one or more context parameters (“context information,” e.g., patient information, clinician information, time information, ambient light information, type of display information) from the time information, and may provide the determined context information to a processing circuitry 227/326 (e.g., at least one of processing circuitry 227 of computing device 216, or processing circuitry 326 of probe 300). The processing circuitry 227/326 may then cause a change in one or more settings of the ultrasound imaging session based on the determined context information.

An clock according to some embodiments may for example include a Quartz Crystal Oscillator (e.g., a circuit that uses a quartz crystal to generate a time signal; a Real-Time Clock (RTC) Circuit (e.g., a digital circuit that keeps track of time using an external crystal oscillator); a Binary-Coded Decimal (BCD) Counter (e.g., a digital circuit that counts in binary-coded decimal format; by dividing the clock frequency by a specific factor, it can be used to generate a time signal); a GPS Clock (e.g., a circuit that uses the Global Positioning System (GPS) to accurately determine the time by receiving signals from GPS satellites and using this information to set its clock; or a 555 timer-based clock (e.g., a circuit that uses a 555 timer as a clock generator to produce square wave pulses of a fixed frequency, which can be counted and used to determine time).

For example, the CIE be configured to process ambient light signals from a CDC that is in the form of one or more light sensors in order to identify ambient light information (e.g., ambient light intensity, and/or whether the display is outdoors or indoors, and/or whether the display is in outdoor in sunlight, outdoor in overcast conditions, etc.) regarding an imaging session to be performed or being performed. The one or more light sensors may be on the probe or on the computing system or both, and may send ambient light signals to the CIE 225. The CIE may for example be configured to determine one or more context parameters (“context information,” e.g., ambient light information, patient information, clinician information, time information, type of display information, location information) from the ambient light information, and may provide the determined context information to a processing circuitry 227/326 (e.g., at least one of processing circuitry 227 of computing device 216, or processing circuitry 326 of probe 300). The processing circuitry 227/326 may then cause a change in one or more settings of the ultrasound imaging session based on the determined context information.

According to an embodiment, the CIE may determine one or more context parameters (“context information,” e.g., patient information, clinician information, ambient light information, ambient light information, type of display information) from the ambient light information, and may provide the determined context information to a processing circuitry 227/326 (e.g., at least one of processing circuitry 227 of computing device 216, or processing circuitry 326 of probe 300). The processing circuitry 227/326 may then cause a change in one or more settings of the ultrasound imaging session based on the determined context information.

A light sensor according to some embodiments may for example include a Photoresistor Circuit. A photoresistor circuit may include a circuit that uses a photoresistor to detect ambient light levels; a photoresistor is a type of resistor that changes its resistance based on the amount of light that hits it; the circuit may include a photoresistor and a fixed resistor, which together form a voltage divider. The output voltage of the circuit is fed to an analog-to-digital converter (ADC) to convert the analog signal into a digital signal that can be processed by a microcontroller.

A light sensor may, according to some embodiments, include a Photodiode Circuit: A photodiode is a type of semiconductor device that converts light into an electrical current. In a photodiode circuit, the photodiode is connected in reverse bias mode, so that the current flow is proportional to the light intensity. The current is then amplified using an operational amplifier (op-amp) and fed to an ADC for further processing.

A light sensor may, according to some embodiments, include a Light-Emitting Diode (LED) Circuit: An LED can also be used as a light sensor. When an LED is exposed to light, it generates a small voltage that can be measured using a voltmeter. The LED can be used in a simple circuit with a fixed resistor and a capacitor to create a low-pass filter that can detect changes in ambient light levels.

A light sensor may, according to some embodiments, include an Ambient Light Sensor (ALS) Circuit: An ALS is a type of integrated circuit that combines a photodiode and an op-amp to create a highly sensitive light sensor. The ALS typically has an integrated ADC and a digital interface, such as I2C, to communicate with a microcontroller.

For example, the CIE be configured to identify display information (e.g., information on one or more displays to be used) regarding an imaging session to be performed or being performed. The CIE may determine the display information from context signals including information on the display, the context signals received at a communication circuitry of the ultrasound imaging system that implements the CIE. The CIE may further identify the display information for example by default (e.g., where it is implemented in circuitry that includes the display, in which case it may have ready access to information regarding the display) or by way of other received information (e.g., other context information, signals received by the CIE allowing the CIE to identify a display being used, such as by way of device pairing information, etc.). The CIE may for example be configured to determine one or more context parameters (“context information,” e.g., ambient light information, patient information, clinician information, time information, type of display information, location information) from display-related data, and may provide the determined context information to a processing circuitry 227/326 (e.g., at least one of processing circuitry 227 of computing device 216, or processing circuitry 326 of probe 300). The processing circuitry 227/326 may then cause a change in one or more settings of the ultrasound imaging session based on the determined context information.

The display information may include type of display. The type of display, according to an embodiment, may include a Liquid Crystal Display (LCD) monitors. These monitors use liquid crystals that are controlled by electric fields to display images and video. They are commonly used in ultrasound imaging and provide high resolution, contrast, and brightness.

The type of display, according to an embodiment, may include a Light Emitting Diode (LED) monitors: These monitors use LEDs to provide backlighting for LCD panels. They are known for their high brightness and energy efficiency, making them well-suited for use in medical imaging.

The type of display, according to an embodiment, may include a Plasma Display Panel (PDP) monitors: PDP monitors use small cells containing plasma to display images and video.

The type of display, according to an embodiment, may include an Organic Light Emitting Diode (OLED) monitors: OLED monitors use organic materials that emit light when an electric current is passed through them. They offer high contrast and deep blacks, making them well-suited for displaying ultrasound images.

Optionally, the CIE may use one or more context parameters to determine another context parameter.

For example, various context parameters, may be used to identify a patient, and to infer at least one of: a history of prior patient imaging sessions, a history of prior patient diagnoses, bodily characteristics of patient (e.g., body mass index (BMI)), patient gender), and, based on one or more of the latter parameters, determine one or more settings of the ultrasound imaging session (e.g., at least one of before or during the imaging session). Determination of the one or more settings may correspond to determination of an imaging session preset.

For example, if the CIE determines that patient X's past three diagnoses have indicated rheumatoid arthritis in the hands, one or more of processing circuitries 227 or 326 are to use such patient information to determine the preset for the imaging session to correspond to a preset for ultrasound imaging of the hands.

For example, if the CIE determines that a BMI of the patient indicates an obese patient, one or more of processing circuitries 227 or 326 are to use such patient information to determine that a frequency setting of the ultrasound imaging probe should correspond to a lower frequency as compared with a frequency setting for a patient with a healthy BMI.

For example, various context parameters, may be used to identify a clinician, and to infer at least one of: a specialty associated with the clinician, a history of prior imaging sessions, a history of prior diagnoses associated with the clinician, and, based on one or more of the latter parameters, determine one or more settings of the ultrasound imaging session (e.g., at least one of before or during the imaging session). Determination of the one or more settings may correspond to determination of an imaging session preset.

For example, if the CIE determines that clinician Y is associated with a cardiology specialty (e.g., because it recognizes the clinician, and/or because it recognizes the location of the imaging as corresponding to the location of a cardiology practice, and/or because it recognizes time information as corresponding to a day and time where a certain clinician or where clinicians of a certain specialty have historically been performing an imaging session or have historically had access to an ultrasound imaging room at a medical facility), one or more of processing circuitries 227 or 326 are to use such clinician information to determine the preset for the imaging session to correspond to a preset for ultrasound imaging of the heart.

For example, various context parameters, may be used to identify a part of the patient body being imaged (e.g., by way of visual information from one or more cameras, by way of words spoken by a clinician or by the patient (e.g., “I will put a gel on your X body part now and it may feel cold.” or “the gel on my X body part feels cold”) by way of a history of presets associated with the clinician and/or with the patient, by way of location associated with a certain specialty, etc.) and, based on one or more of the latter parameters, determine one or more settings of the ultrasound imaging session (e.g., at least one of before or during the imaging session). Determination of the one or more settings may correspond to determination of an imaging session preset.

For example, if the CIE determines that the abdomen is being imaged, one or more of processing circuitries 227 or 326 are to determine the preset for the imaging session to correspond to a preset for ultrasound imaging of the abdomen.

For example, various context parameters, may be used to identify ambient light information (e.g., by way of visual information from one or more cameras, by way of an ambient light sensor, and/or by way of location information and time information, etc.) and, based on one or more of the latter parameters, determine one or more settings of the ultrasound imaging session (e.g., at least one of before or during the imaging session). Determination of the one or more settings may correspond to determination of an imaging session preset.

For example, if the CIE determines that the ambient light corresponds to a sunny outdoor environment, one or more of processing circuitries 227 or 326 are to determine a setting for the display, such as by determining a setting that corresponds to a relatively higher brightness of the display, to a given tilt angle range of the display, and/or to a given color range (e.g., white and/or grey) for the display.

For example, various context parameters may be used to identify display information (e.g., the type of display(s) being used for a given imaging session) and, based on one or more of the latter parameters, determine one or more settings of the ultrasound imaging session (e.g., at least one of before or during the imaging session). Determination of the one or more settings may correspond to determination of an imaging session preset.

For example, if the CIE is between an OLED display and an LCD display, one or more of processing circuitries 227 or 326 are to determine a setting for each of the display types differently.

For example, one or more of processing circuitries 227 or 326 are to determine a contrast setting for each of an OLED display and an LCD display differently. OLED displays have better contrast ratios than LCD displays, which means they can display deeper blacks and brighter whites. This means that when adjusting video signals, the one or more processing circuitries may adjust the contrast levels differently for each display type to achieve optimal image quality.

For example, one or more of processing circuitries 227 or 326 are to determine a color saturation setting for each of an OLED display and an LCD display differently. OLED displays typically have more vivid and saturated colors than LCD displays. This means that when adjusting video signals, the one or more processing circuitries 227 or 326 may need to adjust the color saturation levels differently for each display type to achieve the desired color accuracy.

For example, one or more of processing circuitries 227 or 326 are to determine a brightness setting for each of an OLED display and a LCD display differently. LCD displays tend to be brighter than OLED displays, so the one or more processing circuitries 227 or 326 may need to adjust the brightness levels differently for each display type to achieve optimal image quality.

For example, one or more of processing circuitries 227 or 326 are to determine a gamma setting for each of an OLED display and an LCD display differently Gamma is the measure of how much light a display produces at different levels of input signal. OLED displays tend to have a more linear gamma curve than LCD displays. This means that when adjusting video signals, the one or more processing circuitries 227 or 326 may need to adjust the gamma levels differently for each display type to achieve the desired image quality.

According to an embodiment, after determination by a processing circuitry of the one or more settings (one or more determined settings) based on one or more context parameters as descried above, the processing circuitry may cause to store, in a memory circuitry, a mapping of the one or more determined settings to the one or more context parameters (that were used for the determination of the one or more settings). The memory circuitry may be local (in a same device) to the processing circuitry that is causing to store, or it may be in a different device. In the case of the latter, the processing circuitry may send the mapping for transmission to the memory circuitry. The memory circuitry may correspond to a memory circuitry within a computing node of a cloud computing system, and may be adapted to store multiple mappings of one or more determined settings to corresponding one or more context parameters for a plurality of ultrasound imaging sessions, for example as performed by one or more ultrasound imaging devices over time. In such a case, a determination of the one or more settings by a processing circuitry according to some embodiments may include: accessing a message from the computing node, the message based on mappings stored at the computing node, and determining the one or more settings based on the message.

Computing device 216 may be part of a cloud network, and may further send dynamic information of probe 300 to other nodes of the cloud network. The computing device 216 may include its own I/O circuitry 217 to receive the ultrasound imaging probe signals. The computing device 216 may further include processing circuitry to generate a response signal based on the ultrasound imaging probe signal.

According to some embodiments, once one or more settings of an imaging session have been set, such settings may be adjusted by a clinician, in the event that the settings may not be desirable.

Some embodiments advantageously allow context information of an ultrasound imaging probe to be used for smart preset determination of an ultrasound imaging session. In this manner, a technical performance of the probe and/or of its associated display device can be enhanced by providing a faster implementation of settings, such as presets, and more useful ultrasound images in terms of quality (and hence suitability for more in depth clinical analysis).

Some embodiments advantageously allow an aggregation of a mapping between context information and settings for an individual probe or as between a plurality of probes to allow for adaptive learning in a cloud environment regarding smart and dynamic mapping of context information to settings, such as to presets.

FIG. 4 shows a method 400 to be performed at an ultrasound imaging device according to one embodiment. The method includes, at operation 402, generating, using a context determination circuitry (CDC), and at a beginning of or during an ultrasound imaging session, context signals based on one or more ultrasound imaging session contexts of the ultrasound imaging session; at operation 406, executing a context information engine (CIE) to determine one or more context parameters of the ultrasound imaging session based on the context signals from the CDC; at operation 408, determining one or more ultrasound imaging settings of the ultrasound imaging session based on the one or more context parameters; and at operation 410, causing the one or more settings to be implemented for the ultrasound imaging session

The flows described in FIG. 4 and FIG. 5 are merely representative of operations that may occur in particular embodiments. In other embodiments, additional operations may be performed by the components of the systems shown in FIGS. 1-3. Various embodiments of the present disclosure contemplate any suitable mechanisms for accomplishing the functions described herein. Some of the operations illustrated in FIG. 4 or FIG. 5 may be repeated, combined, modified, or deleted where appropriate. Additionally, operations may be performed in any suitable order without departing from the scope of particular embodiments.

A design may go through various stages, from creation to simulation to fabrication. Data representing a design may represent the design in a number of manners. First, as is useful in simulations, the hardware may be represented using a hardware description language (HDL) or another functional description language. Additionally, a circuit level model with logic and/or transistor gates may be produced at some stages of the design process. Furthermore, most designs, at some stage, reach a level of data representing the physical placement of various devices in the hardware model. In some implementations, such data may be stored in a database file format such as Graphic Data System II (GDS II), Open Artwork System Interchange Standard (OASIS), or similar format.

In any representation of the design, the data may be stored in any form of a machine readable medium. A memory or a magnetic or optical storage such as a disc may be the machine readable medium to store information transmitted via optical or electrical wave modulated or otherwise generated to transmit such information. When an electrical carrier wave indicating or carrying the code or design is transmitted, to the extent that copying, buffering, or re-transmission of the electrical signal is performed, a new copy is made. Thus, a communication provider or a network provider may store on a tangible, machine-readable medium, at least temporarily, an article, such as information encoded into a carrier wave, embodying techniques of embodiments of the present disclosure.

In various embodiments, a medium storing a representation of the design may be provided to a manufacturing system (e.g., a semiconductor manufacturing system capable of manufacturing an integrated circuit and/or related components). The design representation may instruct the system to manufacture a device capable of performing any combination of the functions described above. For example, the design representation may instruct the system regarding which components to manufacture, how the components should be coupled together, where the components should be placed on the device, and/or regarding other suitable specifications regarding the device to be manufactured.

“Circuitry” as used herein may refer to any combination of hardware with software, and/or firmware. As an example, a circuitry includes hardware, such as a micro-controller, associated with a non-transitory medium to store code adapted to be executed by the micro-controller. Therefore, reference to a circuitry, in one embodiment, refers to the hardware, which is specifically configured to recognize and/or execute the code to be held on a non-transitory medium. Furthermore, in another embodiment, use of a circuitry refers to the non-transitory medium including the code, which is specifically adapted to be executed by the microcontroller to perform predetermined operations. And as can be inferred, in yet another embodiment, the term circuitry (in this example) may refer to the combination of the microcontroller and the non-transitory medium. Often circuitry boundaries that are illustrated as separate commonly vary and potentially overlap. For example, a first and a second circuitry may share hardware, software, firmware, or a combination thereof, while potentially retaining some independent hardware, software, or firmware. In one embodiment, use of the term logic includes hardware, such as transistors, registers, or other hardware, such as programmable logic devices.

Logic may be used to implement any of the flows described or functionality of the various components described herein. “Logic” may refer to hardware, firmware, software and/or combinations of each to perform one or more functions. In various embodiments, logic may include a microprocessor or other processing element operable to execute software instructions, discrete logic such as an application-specific integrated circuit (ASIC), a programmed logic device such as a field programmable gate array (FPGA), a storage device containing instructions, combinations of logic devices (e.g., as would be found on a printed circuit board), or other suitable hardware and/or software. Logic may include one or more gates or other circuit components. In some embodiments, logic may also be fully embodied as software. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in storage devices.

Use of the phrase ‘to’ or ‘configured to,’ in one embodiment, refers to arranging, putting together, manufacturing, offering to sell, importing, and/or designing an apparatus, hardware, logic, or element to perform a designated or determined task. In this example, an apparatus or element thereof that is not operating is still ‘configured to’ perform a designated task if it is designed, coupled, and/or interconnected to perform said designated task. As a purely illustrative example, a logic gate may provide a 0 or a 1 during operation. But a logic gate ‘configured to’ provide an enable signal to a clock does not include every potential logic gate that may provide a 1 or 0. Instead, the logic gate is one coupled in some manner that during operation the 1 or 0 output is to enable the clock. Note once again that use of the term ‘configured to’ does not require operation, but instead focuses on the latent state of an apparatus, hardware, and/or element, wherein the latent state the apparatus, hardware, and/or element is designed to perform a particular task when the apparatus, hardware, and/or element is operating.

Furthermore, use of the phrases ‘capable of/to,’ and or ‘operable to,’ in one embodiment, refers to some apparatus, logic, hardware, and/or element designed in such a way to enable use of the apparatus, logic, hardware, and/or element in a specified manner. Note as above that use of to, capable to, or operable to, in one embodiment, refers to the latent state of an apparatus, logic, hardware, and/or element, where the apparatus, logic, hardware, and/or element is not operating but is designed in such a manner to enable use of an apparatus in a specified manner.

The embodiments of methods, hardware, software, firmware, or code set forth above may be implemented via instructions or code stored on a machine-accessible, machine readable, computer accessible, or computer readable medium which are executable by a processing element. A tangible non-transitory machine-accessible/readable medium includes any mechanism that provides (i.e., stores and/or transmits) information in a form readable by a machine, such as a computer or electronic system. For example, a non-transitory machine-accessible medium includes random-access memory (RAM), such as static RAM (SRAM) or dynamic RAM (DRAM); ROM; magnetic or optical storage medium; flash storage devices; electrical storage devices; optical storage devices; acoustical storage devices; other form of storage devices for holding information received from transitory (propagated) signals (e.g., carrier waves, infrared signals, digital signals); etc., which are to be distinguished from the non-transitory mediums that may receive information therefrom.

Instructions used to program logic to perform embodiments of the disclosure may be stored within a memory in the system, such as DRAM, cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, Compact Disc, Read-Only Memory (CD-ROMs), and magneto-optical disks, Read-Only Memory (ROMs), Random Access Memory (RAM), Erasable Programmable Read-Only Memory (EPROM), Electrically Erasable Programmable Read-Only Memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the computer-readable medium includes any type of tangible machine-readable medium suitable for storing or transmitting electronic instructions or information in a form readable by a machine (e.g., a computer).

Some example embodiments will now be described below.

### Examples

Illustrative examples of the technologies disclosed herein are provided below. An embodiment of the technologies may include any one or more, and any combination of, the examples described below.

Example 1 includes an ultrasound imaging system including: context determination circuitry (CDC) to generate context signals based on one or more ultrasound imaging session contexts of an ultrasound imaging session; a memory storing a context information engine (CIE); circuitry coupled to the memory and adapted to execute the CIE to determine one or more context parameters of the ultrasound imaging session based on the context signals from the CDC; and processing circuitry to: access the one or more context parameters before or during the ultrasound imaging session; and determine one or more ultrasound imaging settings of the ultrasound imaging session based on the one or more context parameters; and cause the one or more settings to be implemented for the ultrasound imaging session.

Example 2 includes the subject matter of Example 1, wherein the context signals include at least one of signals corresponding to a measurement of light, signals corresponding to a measurement of sound, signals corresponding to a measurement of radio waves, signals corresponding to a measurement time, signals corresponding to visual signals captured by one or more cameras, or signals corresponding to sound signals captured by one or more microphones, or signals transmitted to the ultrasound imaging system by another system.

Example 3 includes the subject matter of Example 1, wherein the one or more context parameters include one or more of: patient information, clinician information, imaging session location, imaging session time information, information on ambient light at a display location for the ultrasound imaging session (imaging session display location), display device type information, sound information at a location for the ultrasound imaging session (imaging session location), or visual information at the ultrasound imaging session location.

Example 4 includes the subject matter of Example 3, wherein: patient information includes at least one of patient identity, history of prior patient imaging sessions, history of prior patient diagnoses, patient's bodily characteristics, patient age or patient gender; clinician information includes at least one of clinician identity, clinician specialty, or history of clinician settings; imaging session location information may include at least one of an identification of geographic location or an identification of indoor location; information on ambient light at a location where images of the ultrasound imaging session are to be or are being displayed may include at least one of an identification of sunlight or an identification of artificial light; sound information may include at least one of information relating to communication between a clinician and patient, or information spoken by a clinician regarding an imaging session to be performed; and visual information may include an image or a video of at least one of a patient, a clinician, or an imaging session location.

Example 5 includes the subject matter of any one of Examples 1-4, wherein the circuitry is to execute the CIE to determine at least one of the one or more context parameters based on context signals corresponding to respective contexts of the at least one of the one or more context parameters.

Example 6 includes the subject matter of any one of Examples 1-5, wherein the one or more settings include one or more of gain, depth, frequency, time gain compensation, dynamic range, focus, harmonics, mode, focal zone, persistence, automatic gain control, spatial compounding, frequency compounding, sine functions and line density.

Example 7 includes the subject matter of any one of Examples 1-6, wherein the one or more settings correspond to a preset for the ultrasound imaging session.

Example 8 includes the subject matter of Example 7, wherein the preset includes one of: an abdominal preset, a renal preset, a cardiac preset, an obstetrics preset, a musculoskeletal preset, a breast preset, a neonatal preset, an interventional preset, a pelvis preset, or a thyroid preset.

Example 9 includes the subject matter of any one of Examples 1-8, wherein the context signals are first context signals, the one or more ultrasound imaging session contexts are one or more first ultrasound imaging session contexts, the one or more context parameters are one or more first context parameters, and the one or more settings are one or more first settings, wherein: the CDC is to generate, during the ultrasound imaging session and after an implementation of the first settings, second context signals based on one or more second ultrasound imaging session contexts of the ultrasound imaging session; the circuitry coupled to the memory is adapted to execute the CIE to determine one or more second context parameters of the ultrasound imaging session based on the second context signals from the CDC; and the processing circuitry to: access the one or more second context parameters during the ultrasound imaging session; determine one or more second ultrasound imaging settings of the ultrasound imaging session based on the one or more second context parameters; and cause the one or more second settings to be implemented for the ultrasound imaging session.

Example 10 includes the subject matter of any one of Examples 1-9, wherein the memory is a first memory, and the processing circuitry is to cause to store, in a at least one of the first memory or a second memory, a mapping of the one or more settings to the one or more context parameters.

Example 11 includes the subject matter of Example 10, wherein the second memory is in a device different from the ultrasound imaging system, the processing circuitry to send the mapping for transmission to the second memory.

Example 12 includes the subject matter of any one of Examples 1-11, wherein the processing circuitry is to access historical information regarding the one or more context parameters to determine the one or more settings.

Example 13 includes the subject matter of Example 12, wherein the historical information is in a memory of a computing node of a cloud computing system.

Example 14 includes the subject matter of any one of Examples 1-13, including one of an ultrasound imaging probe or a computing system to be paired to an ultrasound imaging probe.

Example 15 includes a method to be performed at an ultrasound imaging system including: generating, using a context determination circuitry (CDC), and at a beginning of or during an ultrasound imaging session, context signals based on one or more ultrasound imaging session contexts of the ultrasound imaging session; executing a context information engine (CIE) to determine one or more context parameters of the ultrasound imaging session based on the context signals from the CDC; determining one or more ultrasound imaging settings of the ultrasound imaging session based on the one or more context parameters; and causing the one or more settings to be implemented for the ultrasound imaging session.

Example 16 includes the subject matter of Example 15, wherein the context signals include at least one of signals corresponding to a measurement of light, signals corresponding to a measurement of sound, signals corresponding to a measurement of radio waves, signals corresponding to a measurement time, signals corresponding to visual signals captured by one or more cameras, or signals corresponding to sound signals captured by one or more microphones, or signals transmitted to the ultrasound imaging system by another system.

Example 17 includes the subject matter of Example 15, wherein the one or more context parameters include one or more of: patient information, clinician information, imaging session location, imaging session time information, information on ambient light at a display location for the ultrasound imaging session, display device type information, sound information at a location for the ultrasound imaging session (imaging session location), or visual information at the ultrasound imaging session location.

Example 18 includes the subject matter of Example 17, wherein: patient information includes at least one of patient identity, history of prior patient imaging sessions, history of prior patient diagnoses, patient's bodily characteristics, patient age or patient gender; clinician information includes at least one of clinician identity, clinician specialty, or history of clinician settings; imaging session location information may include at least one of an identification of geographic location or an identification of indoor location; information on ambient light at a location where images of the ultrasound imaging session are to be or are being displayed may include at least one of an identification of sunlight or an identification of artificial light; sound information may include at least one of information relating to communication between a clinician and patient, or information spoken by a clinician regarding an imaging session to be performed; and visual information may include an image or a video of at least one of a patient, a clinician, or an imaging session location.

Example 19 includes the subject matter of any one of Examples 15-18, executing the CIE to determine at least one of the one or more context parameters is based on context signals corresponding to respective contexts of the at least one of the one or more context parameters.

Example 20 includes the subject matter of any one of Examples 15-19, wherein the one or more settings include one or more of gain, depth, frequency, time gain compensation, dynamic range, focus, harmonics, mode, focal zone, persistence, automatic gain control, spatial compounding, frequency compounding, sine functions and line density.

Example 21 includes the subject matter of any one of Examples 15-20, wherein the one or more settings correspond to a preset for the ultrasound imaging session.

Example 22 includes the subject matter of Example 21, wherein the preset includes one of: an abdominal preset, a renal preset, a cardiac preset, an obstetrics preset, a musculoskeletal preset, a breast preset, a neonatal preset, an interventional preset, a pelvis preset, or a thyroid preset.

Example 23 includes the subject matter of any one of Examples 15-22, wherein the context signals are first context signals, the one or more ultrasound imaging session contexts are one or more first ultrasound imaging session contexts, the one or more context parameters are one or more first context parameters, and the one or more settings are one or more first settings, wherein: generating, using the CDC, during the ultrasound imaging session, and after an implementation of the first settings, second context signals based on one or more second ultrasound imaging session contexts of the ultrasound imaging session; executing the CIE to determine one or more second context parameters of the ultrasound imaging session based on the second context signals from the CDC; determining one or more second ultrasound imaging settings of the ultrasound imaging session based on the one or more second context parameters; and causing the one or more second settings to be implemented for the ultrasound imaging session.

Example 24 includes the subject matter of any one of Examples 15-23, further including causing to store in a memory a mapping of the one or more settings to the one or more context parameters.

Example 25 includes the subject matter of Example 24, wherein the memory is in a device different from the ultrasound imaging system, the method further including sending the mapping for transmission to the memory.

Example 26 includes the subject matter of any one of Examples 15-25, further including accessing historical information regarding the one or more context parameters to determine the one or more settings.

Example 27 includes the subject matter of Example 26, wherein the historical information is in a memory of a computing node of a cloud computing system.

Example 28 includes the subject matter of any one of Examples 15-27, wherein the ultrasound imaging system includes one of an ultrasound imaging probe or a computing system paired to an ultrasound imaging probe.

Example 29 includes a product comprising one or more tangible computer-readable non-transitory storage media comprising computer-executable instructions operable to, when executed by one or more processors of an ultrasound imaging system, cause the one or more processors to implement operations at the ultrasound imaging system, the operations comprising: generating, using a context determination circuitry (CDC), and at a beginning of or during an ultrasound imaging session, context signals based on one or more ultrasound imaging session contexts of the ultrasound imaging session; executing a context information engine (CIE) to determine one or more context parameters of the ultrasound imaging session based on the context signals from the CDC; determining one or more ultrasound imaging settings of the ultrasound imaging session based on the one or more context parameters; and causing the one or more settings to be implemented for the ultrasound imaging session.

Example 30 includes the subject matter of Example 29, wherein the context signals include at least one of signals corresponding to a measurement of light, signals corresponding to a measurement of sound, signals corresponding to a measurement of radio waves, signals corresponding to a measurement time, signals corresponding to visual signals captured by one or more cameras, or signals corresponding to sound signals captured by one or more microphones, or signals transmitted to the ultrasound imaging system by another system.

Example 31 includes the subject matter of Example 29, wherein the one or more context parameters include one or more of: patient information, clinician information, imaging session location, imaging session time information, information on ambient light at a display location for the ultrasound imaging session, display device type information, sound information at a location for the ultrasound imaging session (imaging session location), or visual information at the ultrasound imaging session location.

Example 32 includes the subject matter of Example 31, wherein: patient information includes at least one of patient identity, history of prior patient imaging sessions, history of prior patient diagnoses, patient's bodily characteristics, patient age or patient gender; clinician information includes at least one of clinician identity, clinician specialty, or history of clinician settings; imaging session location information may include at least one of an identification of geographic location or an identification of indoor location; information on ambient light at a location where images of the ultrasound imaging session are to be or are being displayed may include at least one of an identification of sunlight or an identification of artificial light; sound information may include at least one of information relating to communication between a clinician and patient, or information spoken by a clinician regarding an imaging session to be performed; and visual information may include an image or a video of at least one of a patient, a clinician, or an imaging session location.

Example 33 includes the subject matter of any one of Examples 29-32, executing the CIE to determine at least one of the one or more context parameters is based on context signals corresponding to respective contexts of the at least one of the one or more context parameters.

Example 34 includes the subject matter of any one of Examples 29-33, wherein the one or more settings include one or more of gain, depth, frequency, time gain compensation, dynamic range, focus, harmonics, mode, focal zone, persistence, automatic gain control, spatial compounding, frequency compounding, sine functions and line density.

Example 35 includes the subject matter of any one of Examples 29-34, wherein the one or more settings correspond to a preset for the ultrasound imaging session.

Example 36 includes the subject matter of Example 35, wherein the preset includes one of: an abdominal preset, a renal preset, a cardiac preset, an obstetrics preset, a musculoskeletal preset, a breast preset, a neonatal preset, an interventional preset, a pelvis preset, or a thyroid preset.

Example 37 includes the subject matter of any one of Examples 29-36, wherein the context signals are first context signals, the one or more ultrasound imaging session contexts are one or more first ultrasound imaging session contexts, the one or more context parameters are one or more first context parameters, and the one or more settings are one or more first settings, wherein: generating, using the CDC, during the ultrasound imaging session, and after an implementation of the first settings, second context signals based on one or more second ultrasound imaging session contexts of the ultrasound imaging session; executing the CIE to determine one or more second context parameters of the ultrasound imaging session based on the second context signals from the CDC; determining one or more second ultrasound imaging settings of the ultrasound imaging session based on the one or more second context parameters; and causing the one or more second settings to be implemented for the ultrasound imaging session.

Example 38 includes the subject matter of any one of Examples 29-37, the operations further including causing to store in a memory a mapping of the one or more settings to the one or more context parameters.

Example 39 includes the subject matter of Example 38, wherein the memory is in a device different from the ultrasound imaging system, the operations further including sending the mapping for transmission to the memory.

Example 40 includes the subject matter of any one of Examples 29-39, the operations further including accessing historical information regarding the one or more context parameters to determine the one or more settings.

Example 41 includes the subject matter of Example 40, wherein the historical information is in a memory of a computing node of a cloud computing system.

Example 42 includes the subject matter of any one of Examples 29-41, wherein the ultrasound imaging system includes one of an ultrasound imaging probe or a computing system to be paired to an ultrasound imaging probe.

Example 43 includes one or more computer-readable media comprising instructions stored thereon that, when executed, cause one or more processors to perform the method of any one of Examples 15-28.

Example 69 includes an imaging device comprising the apparatus of any one of Examples 15-28, and further including the user interface device.

Example 70 includes a product comprising one or more tangible computer-readable non-transitory storage media comprising computer-executable instructions operable to, when executed by at least one computer processor, enable the at least one processor to perform the method of any one of Examples 15-28.

Example 71 includes means for carrying out the methods of any one of Examples 15-18.

What is claimed is:

1. An ultrasound imaging system including:
context determination circuitry (CDC) to generate context signals based on one or more ultrasound imaging session contexts of an ultrasound imaging session;
a memory storing a context information engine (CIE);
circuitry coupled to the memory and adapted to execute the CIE to determine one or more context parameters of the ultrasound imaging session based on the context signals from the CDC; and
processing circuitry to:
access the one or more context parameters before or during the ultrasound imaging session; and
determine one or more ultrasound imaging settings of the ultrasound imaging session based on the one or more context parameters; and
cause the one or more settings to be implemented for the ultrasound imaging session.

2. The ultrasound imaging system of claim 1, wherein the context signals include at least one of signals corresponding to a measurement of light, signals corresponding to a measurement of sound, signals corresponding to a measurement of radio waves, signals corresponding to a measurement time, signals corresponding to visual signals captured by one or more cameras, or signals corresponding to sound signals captured by one or more microphones, or signals transmitted to the ultrasound imaging system by another system.

3. The ultrasound imaging system of claim 1, wherein the one or more context parameters include one or more of: patient information, clinician information, imaging session location, imaging session time information, information on ambient light at a display location for the ultrasound imaging session (imaging session display location), display device type information, sound information at a location for the ultrasound imaging session (imaging session location), or visual information at the ultrasound imaging session location.

4. The ultrasound imaging system of claim 3, wherein:
patient information includes at least one of patient identity, history of prior patient imaging sessions, history of prior patient diagnoses, patient's bodily characteristics, patient age or patient gender;
clinician information includes at least one of clinician identity, clinician specialty, or history of clinician settings;
imaging session location information may include at least one of an identification of geographic location or an identification of indoor location;
information on ambient light at a location where images of the ultrasound imaging session are to be or are being displayed may include at least one of an identification of sunlight or an identification of artificial light;
sound information may include at least one of information relating to communication between a clinician and patient, or information spoken by a clinician regarding an imaging session to be performed; and
visual information may include an image or a video of at least one of a patient, a clinician, or an imaging session location.

5. The ultrasound imaging system of claim 1, wherein the circuitry is to execute the CIE to determine at least one of the one or more context parameters based on context signals corresponding to respective contexts of the at least one of the one or more context parameters.

6. The ultrasound imaging system of claim 1, wherein the one or more settings include one or more of gain, depth, frequency, time gain compensation, dynamic range, focus, harmonics, mode, focal zone, persistence, automatic gain control, spatial compounding, frequency compounding, sine functions and line density.

7. The ultrasound imaging system of claim 1, wherein the one or more settings correspond to a preset for the ultrasound imaging session.

8. The ultrasound imaging system of claim 7, wherein the preset includes one of: an abdominal preset, a renal preset, a cardiac preset, an obstetrics preset, a musculoskeletal preset, a breast preset, a neonatal preset, an interventional preset, a pelvis preset, or a thyroid preset.

9. The ultrasound imaging system of claim 1, wherein the context signals are first context signals, the one or more ultrasound imaging session contexts are one or more first ultrasound imaging session contexts, the one or more context parameters are one or more first context parameters; and the one or more settings are one or more first settings, wherein:
the CDC is to generate, during the ultrasound imaging session and after an implementation of the first settings, second context signals based on one or more second ultrasound imaging session contexts of the ultrasound imaging session;
the circuitry coupled to the memory is adapted to execute the CIE to determine one or more second context parameters of the ultrasound imaging session based on the second context signals from the CDC; and
the processing circuitry to:
access the one or more second context parameters during the ultrasound imaging session;
determine one or more second ultrasound imaging settings of the ultrasound imaging session based on the one or more second context parameters; and
cause the one or more second settings to be implemented for the ultrasound imaging session.

10. The ultrasound imaging system of claim 1, wherein the memory is a first memory, and the processing circuitry is to cause to store, in a at least one of the first memory or a second memory, a mapping of the one or more settings to the one or more context parameters.

11. The ultrasound imaging system of claim 10, wherein the second memory is in a device different from the ultrasound imaging system, the processing circuitry to send the mapping for transmission to the second memory.

12. The ultrasound imaging system of claim 11, wherein the processing circuitry is to access historical information regarding the one or more context parameters to determine the one or more settings.

13. The ultrasound imaging system of claim 12, wherein the historical information is in a memory of a computing node of a cloud computing system.

14. The ultrasound imaging system of claim 13, including one of an ultrasound imaging probe or a computing system to be paired to an ultrasound imaging probe.

15. A product comprising one or more tangible computer-readable non-transitory storage media comprising computer-executable instructions operable to, when executed by one or more processors of an ultrasound imaging system, cause the one or more processors to implement operations at the ultrasound imaging system, the operations comprising:
generating, using a context determination circuitry (CDC), and at a beginning of or during an ultrasound imaging session, context signals based on one or more ultrasound imaging session contexts of the ultrasound imaging session;
executing a context information engine (CIE) to determine one or more context parameters of the ultrasound imaging session based on the context signals from the CDC;
determining one or more ultrasound imaging settings of the ultrasound imaging session based on the one or more context parameters; and
causing the one or more settings to be implemented for the ultrasound imaging session.

16. The product of claim 15, wherein the context signals include at least one of signals corresponding to a measurement of light, signals corresponding to a measurement of sound, signals corresponding to a measurement of radio waves, signals corresponding to a measurement time, signals corresponding to visual signals captured by one or more cameras, or signals corresponding to sound signals captured by one or more microphones, or signals transmitted to the ultrasound imaging system by another system.

17. The product of claim 15, wherein the one or more context parameters include one or more of: patient information, clinician information, imaging session location, imaging session time information, information on ambient light at a display location for the ultrasound imaging session, display device type information, sound information at a location for the ultrasound imaging session (imaging session location), or visual information at the ultrasound imaging session location.

18. The product of claim 17, wherein:
patient information includes at least one of patient identity, history of prior patient imaging sessions, history of prior patient diagnoses, patient's bodily characteristics, patient age or patient gender;
clinician information includes at least one of clinician identity, clinician specialty, or history of clinician settings;
imaging session location information may include at least one of an identification of geographic location or an identification of indoor location;
information on ambient light at a location where images of the ultrasound imaging session are to be or are being displayed may include at least one of an identification of sunlight or an identification of artificial light;
sound information may include at least one of information relating to communication between a clinician and patient, or information spoken by a clinician regarding an imaging session to be performed; and
visual information may include an image or a video of at least one of a patient, a clinician, or an imaging session location.

19. The product of claim 18, wherein executing the CIE to determine at least one of the one or more context parameters is based on context signals corresponding to respective contexts of the at least one of the one or more context parameters.

20. The product of claim 15, wherein the one or more settings include one or more of gain, depth, frequency, time gain compensation, dynamic range, focus, harmonics, mode, focal zone, persistence, automatic gain control, spatial compounding, frequency compounding, sine functions and line density.

21. A method to be performed at an ultrasound imaging system including:
generating, using a context determination circuitry (CDC), and at a beginning of or during an ultrasound imaging session, context signals based on one or more ultrasound imaging session contexts of the ultrasound imaging session;
executing a context information engine (CIE) to determine one or more context parameters of the ultrasound imaging session based on the context signals from the CDC;
determining one or more ultrasound imaging settings of the ultrasound imaging session based on the one or more context parameters; and
causing the one or more settings to be implemented for the ultrasound imaging session.

22. The method of claim 21, wherein the context signals include at least one of signals corresponding to a measurement of light, signals corresponding to a measurement of sound, signals corresponding to a measurement of radio waves, signals corresponding to a measurement time, signals corresponding to visual signals captured by one or more cameras, or signals corresponding to sound signals captured by one or more microphones, or signals transmitted to the ultrasound imaging system by another system.

23. The method of claim 21, wherein the one or more context parameters include one or more of: patient information, clinician information, imaging session location, imaging session time information, information on ambient light at a display location for the ultrasound imaging session, display device type information, sound information at a location for the ultrasound imaging session (imaging session location), or visual information at the ultrasound imaging session location.

24. The method of claim 23, wherein:
patient information includes at least one of patient identity, history of prior patient imaging sessions, history of prior patient diagnoses, patient's bodily characteristics, patient age or patient gender;
clinician information includes at least one of clinician identity, clinician specialty, or history of clinician settings;
imaging session location information may include at least one of an identification of geographic location or an identification of indoor location;
information on ambient light at a location where images of the ultrasound imaging session are to be or are being displayed may include at least one of an identification of sunlight or an identification of artificial light;
sound information may include at least one of information relating to communication between a clinician and patient, or information spoken by a clinician regarding an imaging session to be performed; and
visual information may include an image or a video of at least one of a patient, a clinician, or an imaging session location.

25. The method of claim 24, executing the CIE to determine at least one of the one or more context parameters is based on context signals corresponding to respective contexts of the at least one of the one or more context parameters.
